(19) United States
(12) Patent Application Publication
(10) Pub. No.: US 2008/0051352 A1
(43) Pub. Date: Feb. 28, 2008

(54) Novel process for the preparation of telithromycin

(75) Inventors: Pandurang Balwant Deshpande, Vadodara (IN); Manish Kanchanbhai Patel, Vadodara (IN); Kalpesh Haribhai Dhameliya, Vadodara (IN); Parven Kumar Luthra, Vadodara (IN)

Correspondence Address: HAMRE, SCHUMANN, MUELLER & LARSON, P.C., P.O. BOX 2902, MINNEAPOLIS, MN, 55402-0902, US

(73) Assignee: ALEMBIC LIMITED, Vadodara (IN)

(21) Appl. No.: 11/640,675

(22) Filed: Dec. 18, 2006

(30) Foreign Application Priority Data
Aug. 25, 2006 (IN) 1347/MUM/2006

(51) Int. Cl. A61K 31/7052; C07H 17/08

(52) U.S. Cl. 514/28; 536/7.4

(57) ABSTRACT

The present invention relates to the process for the preparation of compounds of formula (I) or its pharmaceutically acceptable salts

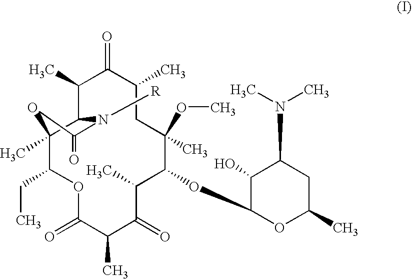

wherein, R is

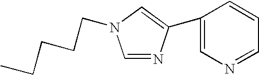

### FIELD OF THE INVENTION

The present invention relates to the process for the preparation of Telithromycin of formula (I) or pharmaceutically acceptable salts thereof. It also provides novel intermediates and process for the preparation of the same.

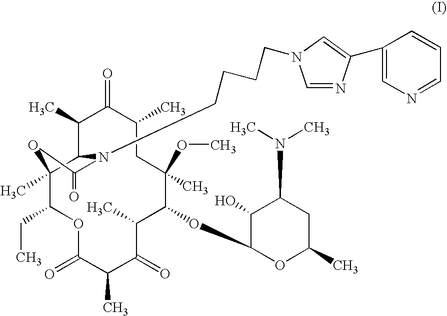

Telithromycin of formula (I) is know to have antibiotic activity.

### BACKGROUND OF THE INVENTION

Macrolide compounds are known for anti bacterial activity. The rapid development of antibiotic resistance among the major respiratory pathogens has created a serious problem for the effective management of respiratory tract infections. There is a great medical need for new antibiotics to address the problem of antibiotic resistance. Under these circumstances, several novel series of macrolides with a common C-3 ketone group were recently introduced, which are collectively known as ketolides.

Ketolides represent a novel class of macrolide antibiotics that have received much attention recently on account of their excellent activity against resistant organisms. Most ketolides are derivatives of erythromycin, a potent and safe antibiotic widely prescribed for the treatment of respiratory tract infections for more than four decades. Ketolides are 14-membered ring macrolide derivatives characterized by a keto group at the C-3 position [Curr. Med. Chem.—Anti-Infective Agents, 2002, 1, 15-34]. Several Ketolide compounds are under clinical investigation. However, Telithromycin of Formula (I) is the first agent to receive approvable status in this class of drugs.

U.S. Pat. No. 5,635,485 discloses several ketolide compounds, which are prepared by condensing compounds of formula (II) with amine of formula (III) in a solvent for prolonged hours to yield compound of formula (IV), followed by removal of protecting group Z′ at 2′ position by hydrolysis as shown in SCHEME-1. Furthermore, formula (II) is prepared by following the process disclosed in U.S. Pat. No. 5,527,780.

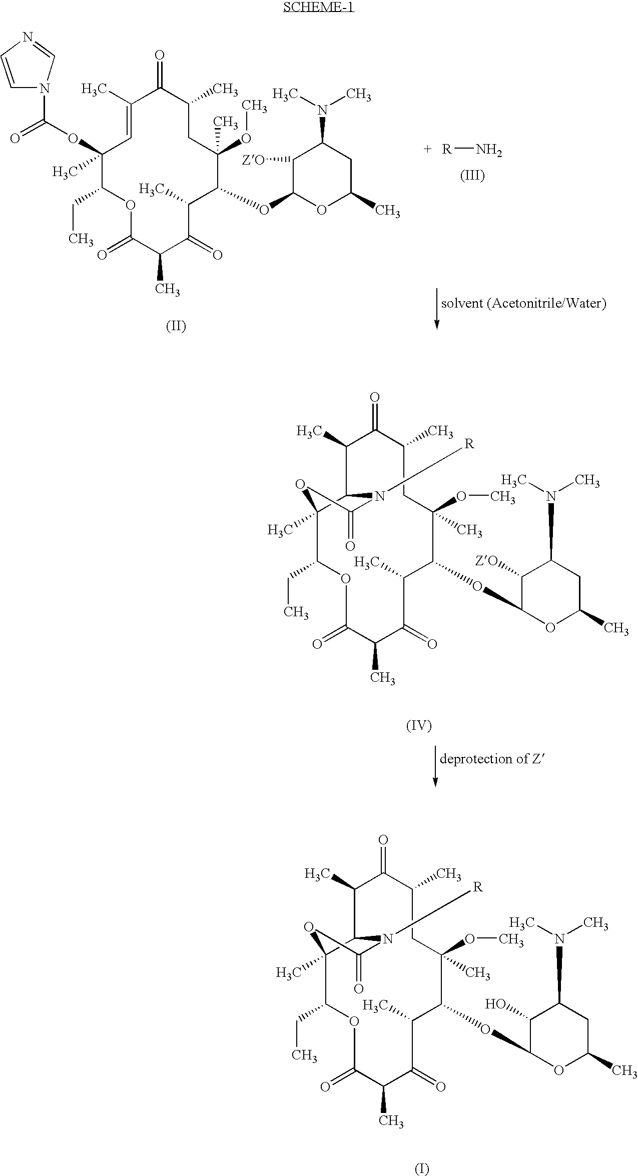

wherein, definition of R and Z′ is as described in above referred patent. Accordingly, Telithromycin is prepared by condensing compound of formula (II) with amine of formula (III), wherein

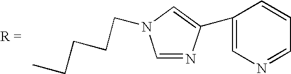

followed by removal of the protecting group to yield Telithromycin of formula (I).

The process described in U.S. Pat. No. 5,635,485 suffers several drawbacks like:
(i) Condensation of compound of formula (II) with formula compound of (III) is cumbersome. Moreover it is very difficult to remove unreacted reagents and impurities formed during the reaction.
(ii) The isolation and purification of the desired compound of formula (I) cannot be done without laborious column chromatography, which is operationally difficult at commercial production level.

Current Medicinal Chemistry, 2001, Vol. 8, 1727-1758 also describes the process for the preparation of various ketolides, including Telithromycin in which Clarithromycin (formula V) is reacted with hydrochloric acid to remove cladinose ring at C-3 position (formula VI) followed by selective acetylation of the 2′-hydroxy group in formula VI and selective oxidation of the 3-hydroxy group generated ketolide of formula VII. Further, 11-hydorxy group of compound of formula (VII) is selectively mesylated followed by base induced β-elimination to furnish α,β-unsaturated ketone (formula VIII). The compound of formula (VIII) is further treated with sodium hydride and carbonyldiimidazole to form 12-O-acyl imidazole of formula (II), which upon stereoselective cyclization with (4-(3-pyridinyl)-imidazol-1-yl)-butylamine and subsequent deprotection of the 2′-hydroxy group gives Telithromycin of Formula (I). This process is outlined in following SCHEME-2

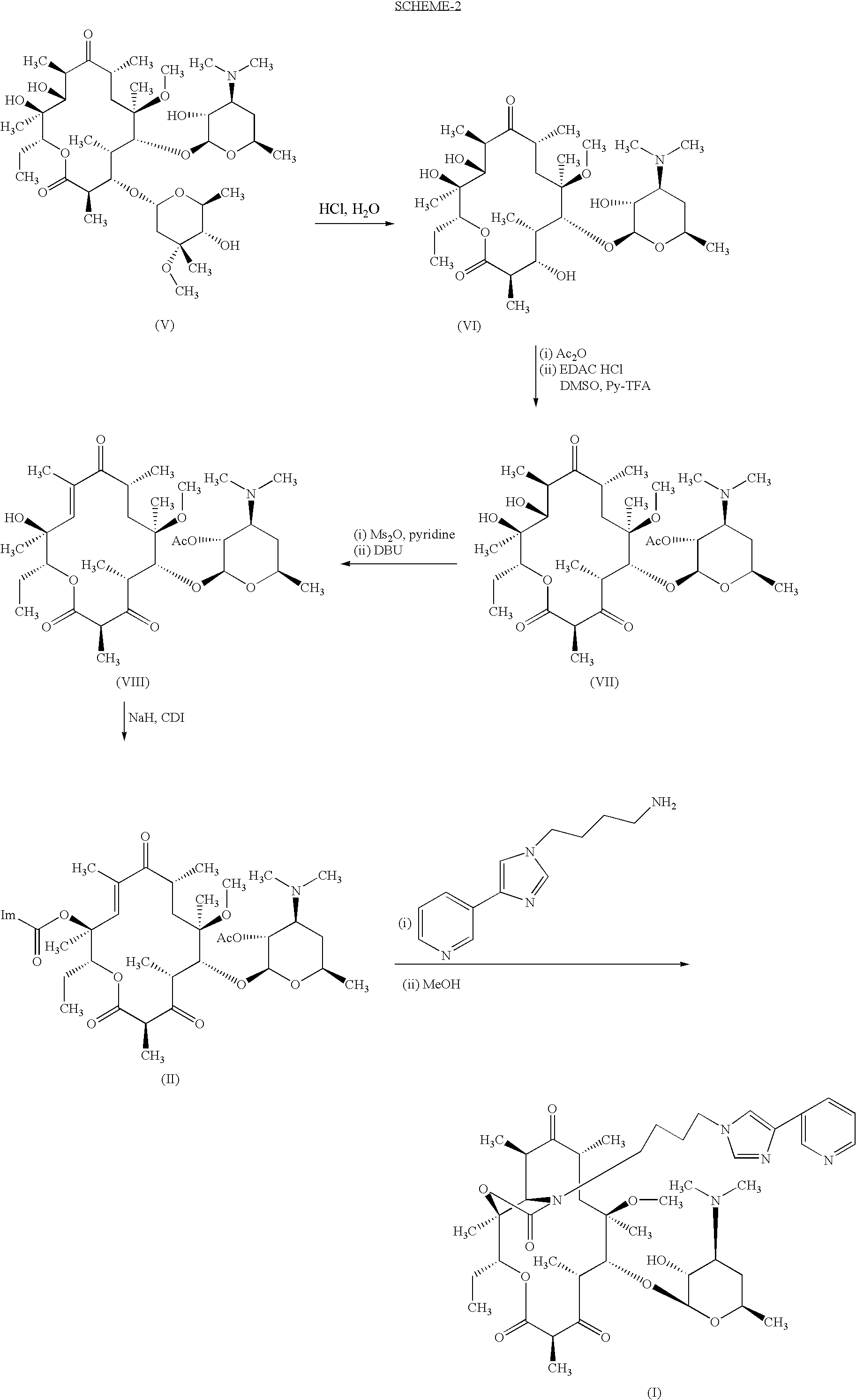

However, this process also suffers from similar drawbacks as listed above. Moreover the use of NaH is hazardous and extremely difficult to handle at the plant scale due to its pyrophoric nature.

In light of the above difficulties for the preparation of Telithromycin, there exists a need to develop a process which is suitable for large scale production.

### OBJECTS OF THE INVENTION

It is therefore an object of present invention to provide a process for the preparation of Telithromycin of formula (I) or pharmaceutically acceptable salts thereof.

Another object of the present invention is to provide a process for the preparation of Telithromycin, which would be high yielding, cost effective, easy to operate at industrial scale and would not involve the use of moisture sensitive, pyrophoric compounds like sodium hydride.

A further objective of the invention is to provide a process of manufacture of Telithromycin that would involve selective mild reaction conditions.

A further object of the invention is to provide a process of manufacture of Telithromycin that would be industrially feasible.

Yet, another object of the present invention is to provide a novel compound of formula (X) which is an useful intermediate for the preparation of Telithromycin of formula (I) or pharmaceutically acceptable salts thereof.

### SUMMARY OF THE INVENTION

In accordance with the object of the present invention, one aspect provides the process for the preparation of Telithromycin of formula (I) or its pharmaceutically acceptable salts

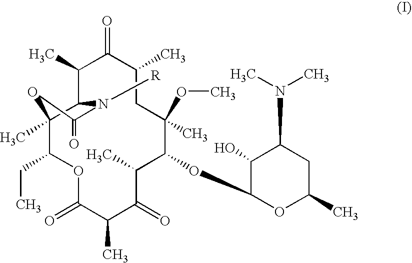

wherein R is

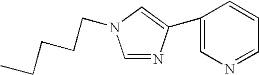

comprises steps of,
(a) reacting compound of formula (IX) with compound of formula (A),

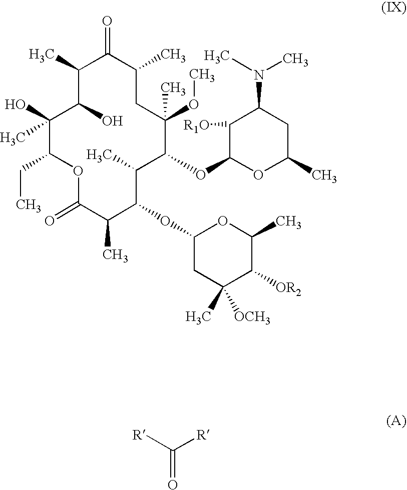

wherein R′ is selected from group comprising of methoxy, ethoxy, propoxy, pentamethylene, piperidinyl, 1-methyl imidazolyl and the like, in the presence of polar solvent and a base to obtain the compound of formula (X)

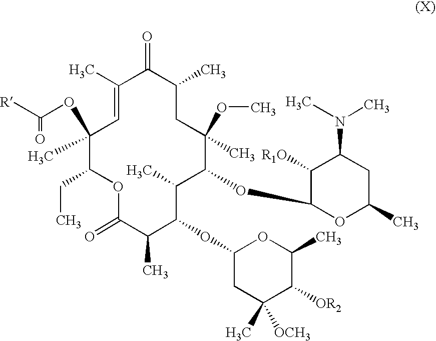

wherein R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴ and R⁵ is other than hydrogen atom) or

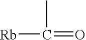

or (wherein R_{b} is an alkyl group having 1 to 10 carbon atoms or a substituted or unsubstituted aryl)
(b) condensing compound of formula (X) with R—NH₂ in a suitable polar solvent to obtain compound of formula (XI)

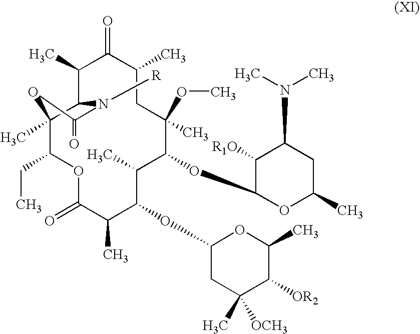

wherein R, R₁ and R₂ are same as defined hereinabove.
(c) treating compound of formula (XI) with an acid to obtain compound of formula (XII)

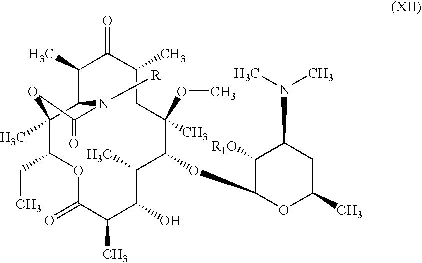

(d) oxidising compound of formula (XII) in the presence of an oxidizing agent to give compound of formula (XIII)

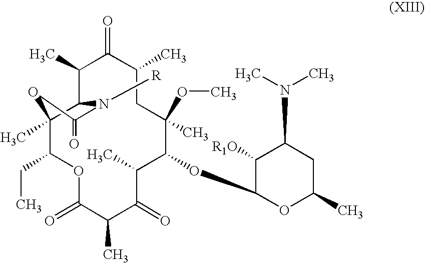

(e) treating compound of formula (XIII) with an alcohol.

The synthetic scheme for the preparation of Telithromycin of formula (I) or its pharmaceutically acceptable salts is as shown in SCHEME-3:

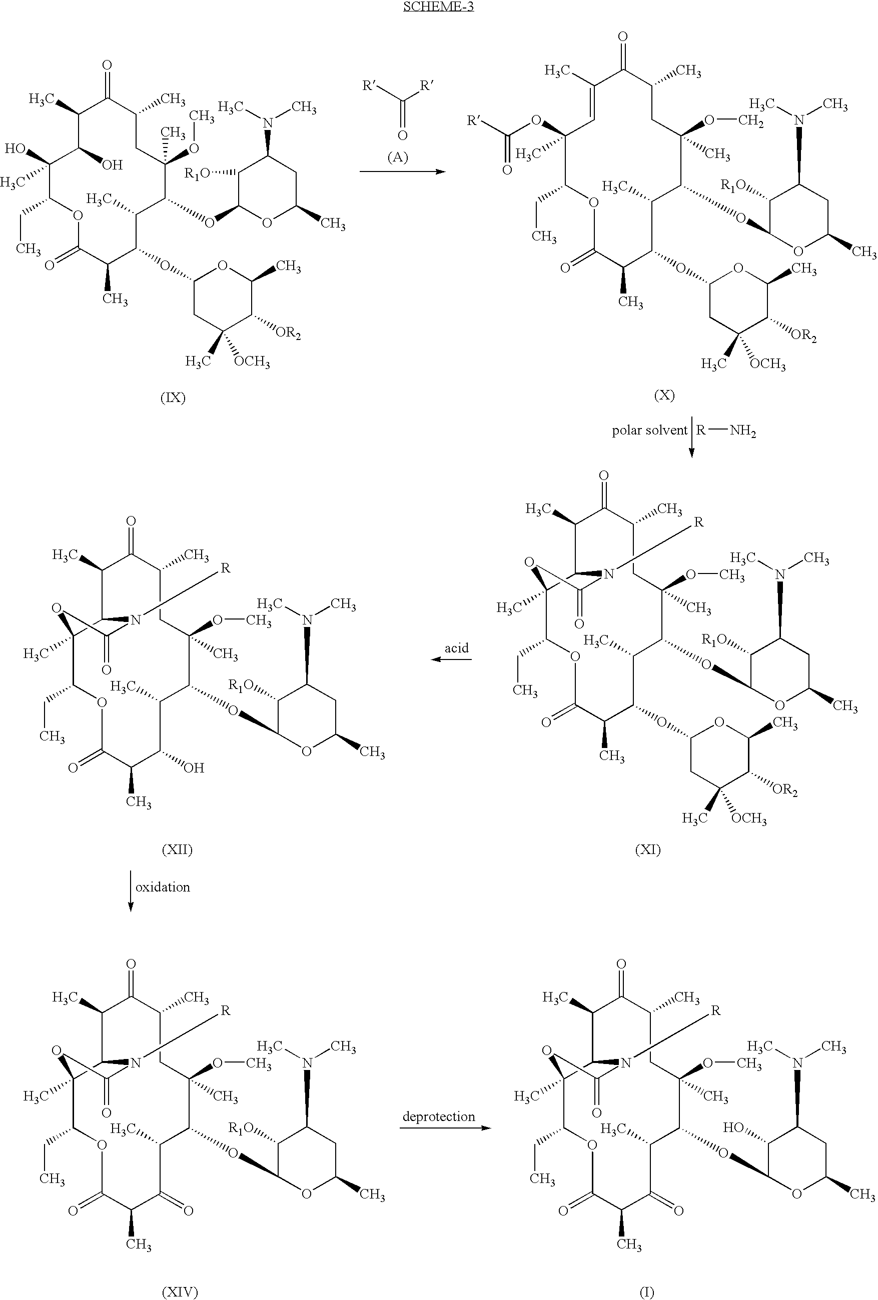

Further aspect of the present invention provides an alternative process for the preparation of Telithromycin of formula (I) or its pharmaceutically acceptable salts, comprises steps of,
(a) reacting compound of formula (IX) with compound of formula (A),

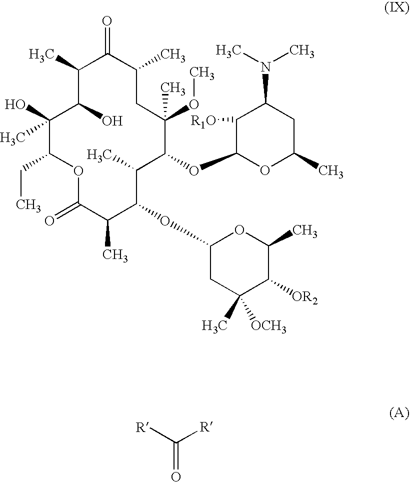

wherein R′ is selected from group comprising of methoxy, ethoxy, propoxy, pentamethylene, piperidinyl, 1-methyl imidazolyl and the like, in the presence of polar solvent and a base to obtain the compound of formula (X)

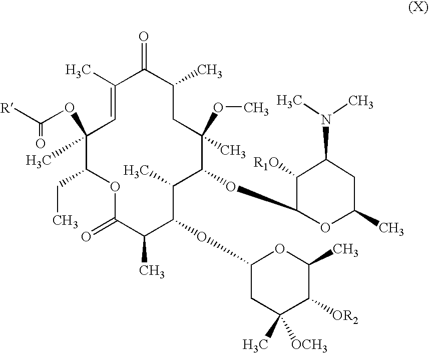

wherein R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴ and R⁵ is other than hydrogen atom) or

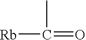

(wherein R_{b} is an alkyl group having 1 to 10 carbon atoms or a substituted or unsubstituted aryl)
(b) condensing compound of formula (X) with R—NH₂ in a suitable polar solvent to give compound of formula (XI).

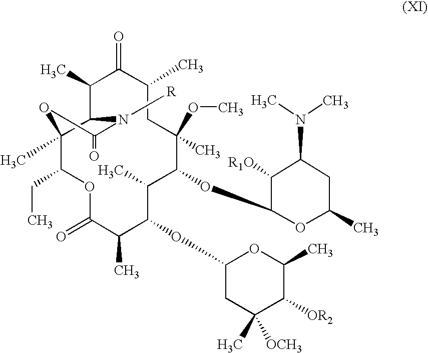

wherein R, R₁ and R₂ are same as defined hereinabove
(c) treating the compound of formula (XI) with an acid to obtain compound of formula (XII)

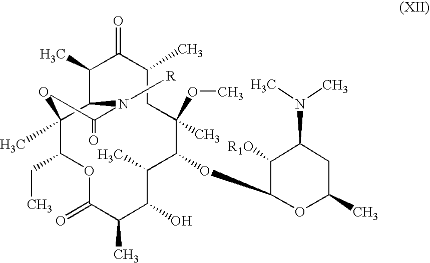

(f) treating compound of formula (XII) with an alcohol to obtain compound of formula (XIV)

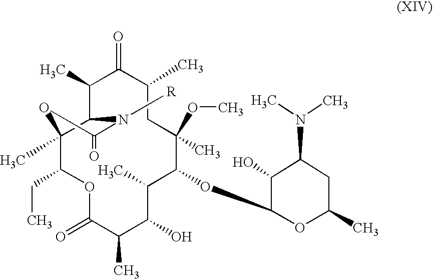

(g) selectively oxidizing compound of formula (XIV) in the presence of an oxidizing agent to form desired ketolide compound of formula (I)

The synthetic scheme of the alternate process is represented in SCHEME-4:

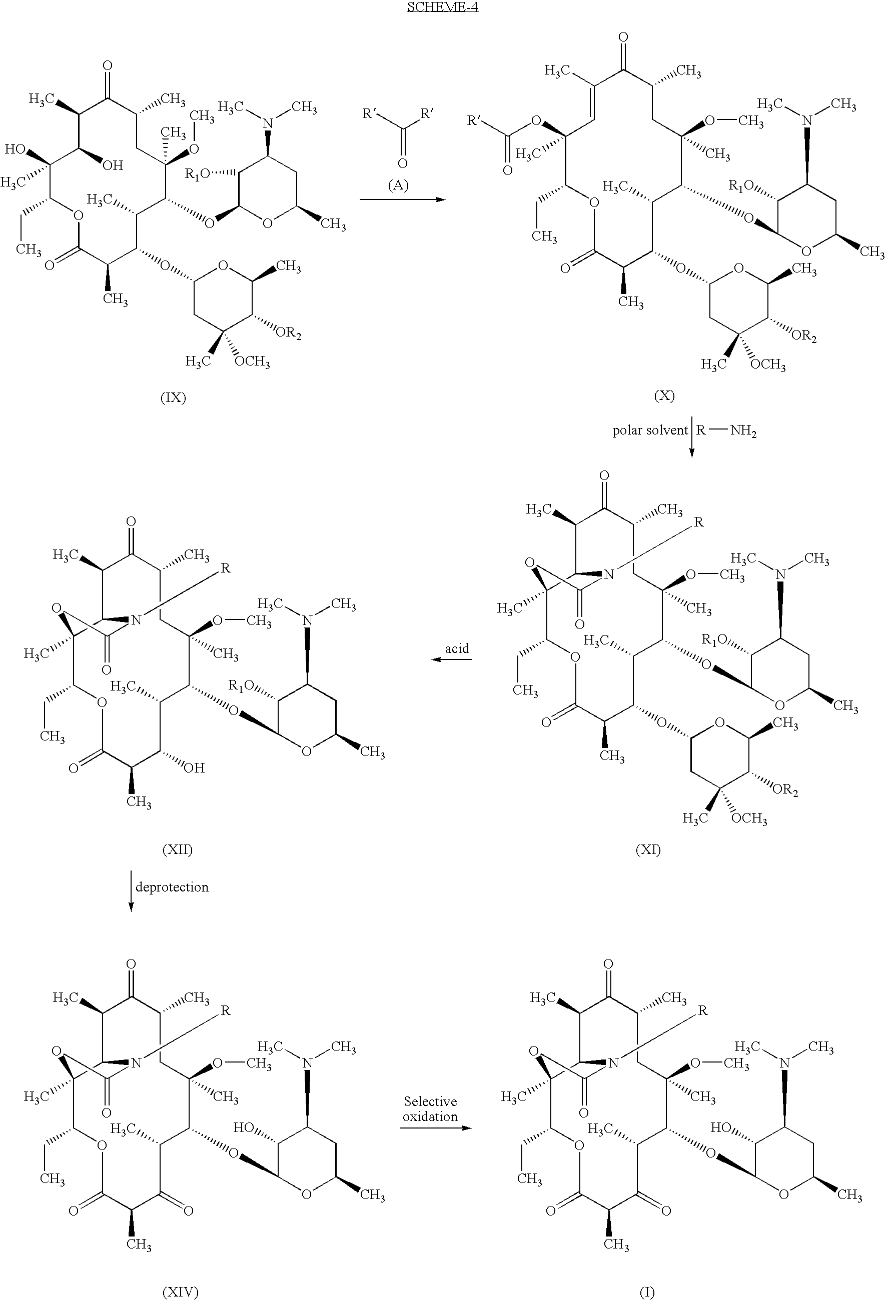

Yet another aspect of the present invention provides novel compound of formula (X).

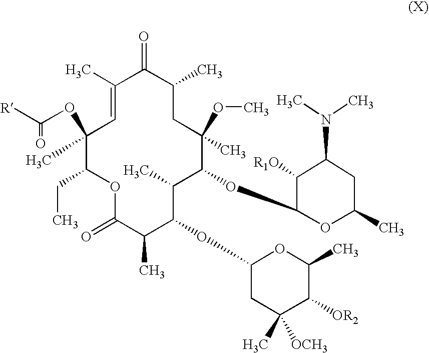

wherein R′ is selected from group comprising of methoxy, ethoxy, propoxy, pentamethylene, piperidinyl, 1-methyl imidazolyl and the like and R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴ and R⁵ is other than hydrogen atom) or

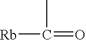

(wherein R_{b} is an alkyl group having 1 to 10 carbon atoms or a substituted or unsubstituted aryl)

Further aspect of present invention provides process for the preparation of novel compound of formula (X) comprising of reacting compound of formula (IX) with compound of formula (A),

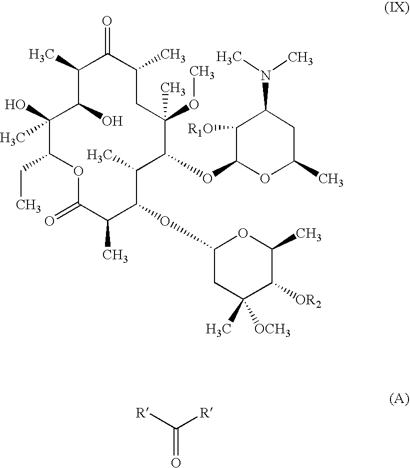

wherein R′ is selected from group comprising of methoxy, ethoxy, propoxy, pentamethylene, piperidinyl, 1-methyl imidazolyl and the like, in the presence of a polar solvent and base to obtain the compound of formula (X)

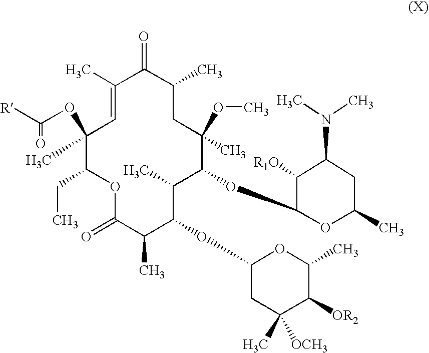

wherein R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴ and R⁵ is other than hydrogen atom) or

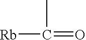

(wherein R_{b} is an alkyl group having 1 to 10 carbon atoms or a substituted or unsubstituted aryl)

### DETAILED DESCRIPTION OF THE INVENTION

The term “treating” as used hereinabove refers to simple dictionary meaning: “To subject to a process, action, or change, especially to a chemical or physical process or application”.

The present invention provides process for the preparation of Telithromycin of formula (I) or pharmaceutically acceptable salts thereof, comprises steps of,
(a) reacting compound of formula (IX) with compound of formula (A),

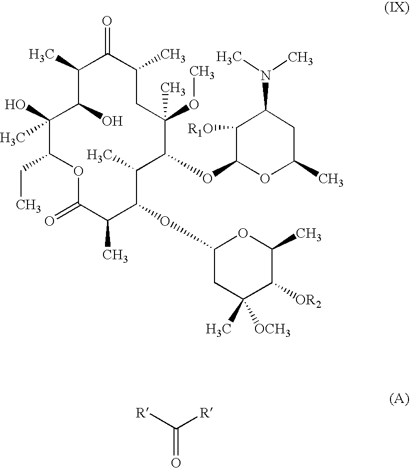

wherein R′ is selected from group comprising of methoxy, ethoxy, propoxy, pentamethylene, piperidinyl, 1-methyl imidazolyl and the like, in the presence of polar solvent and a base to obtain compound of formula (X)

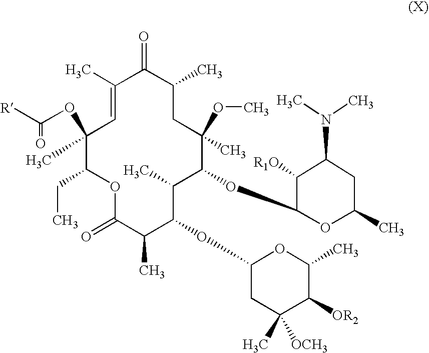

wherein R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴ and R⁵ is other than hydrogen atom) or

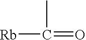

(wherein R_{b} is an alkyl group having 1 to 10 carbon atoms or a substituted or unsubstituted aryl)
(b) condensing compound of formula (X) with R—NH₂ in a suitable polar solvent to obtain compound of formula (XI)

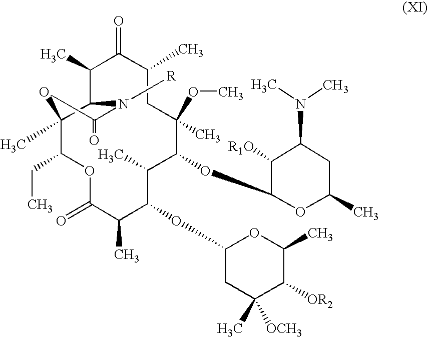

wherein R, R₁ and R₂ are same as defined hereinabove
(c) treating compound formula (XI) with an acid to obtain compound of formula (XII)

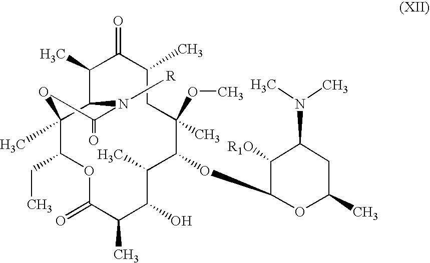

(d) oxidizing compound of formula (XII) in the presence of an oxidizing agent to form compound of formula (XIII)

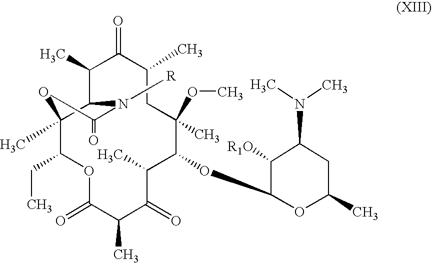

(e) treating compound of formula (XIII) with an alcohol.

The polar solvent use in step (a) is selected from dimethylformamide, tetrahydrofuran, acetonitrile and the like or mixtures thereof. The base used in step (a) is selected from DBU, Triethylamine and diisopropylethylamine.

Non-limiting examples of compound of formula (A) are 1,1′-carbonylbis(2-methylimidazole), 1,1′-carbonyldipiperidine, bis(pentamethylene)urea, dimethylcarbonyl, diethoxycarbonyl, dipropoxycarbonyl and the like.

The examples of silyl protecting group includes but are not limited to trimethylsilyl group, a triethylsilyl group, an isopropyldimethylsilyl group, a tert-butyldimethylsilyl group, a (triphenylmethyl)dimethylsilyl group, a tert-butyldiphenylsilyl group, a diphenylmethylsilyl group, diphenylvinylsilyl group, a methyldiisopropylsilyl group, a tribenzylsilyl group, a tri(p-xylyl)silyl group, a triphenylsilyl group, a diphenylsilyl group, a dimethyloctadecylsilyl group and the like. The examples of an alkyl group having 1 to 10 carbon atoms or a substituted or unsubstituted aryl includes but are not limited to acetyl group, benzyloxy carbonyl group or benzoyl group.

The polar solvent used in step (b) is either polar aprotic solvent or polar protic solvent. The examples of which includes but are not limited to methanol, ethanol, isopropanol, n-propanol, n-butanol, iso butyl alcohol, tert-butyl alcohol, methoxyethanol, ethoxyethanol, pentanol, neo-pentyl alcohol, t-pentyl alcohol, cyclohexanol, ethylene glycol, propylene glycol, benzyl alcohol, phenol, glycerol, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethylsulfoxide, propionitrile, ethyl formate, methyl acetate, hexachloroacetone, HMPA, HMPT, acetone, ethyl methyl ketone, ethyl acetate, isopropyl acetate, t-butyl acetate, sulfolane, N,N-dimethylpropionamide, nitromethane, nitrobenzene, teteahydrofuran (THF), dioxane, water, polyethers and the like or mixtures thereof, preferably dimethylfomamide and acetonitrile.

The reaction step (b) is carried out at a temperature of 0° to boiling temperature of the solvent and preferably at 200 to 160° C. This step can be optionally carried out in the presence of base selected from DBU, triethylamine and di isopropylethylamine.

Acid as referred in step (c) is organic acid or inorganic acid selected from group comprising of hydrochloric acid, hydrobromic acid, sulfuric acid, nitric acid, phosphoric acid, perchloric acid or hydrofluoric acid. The preferred acid is hydrochloric acid. The reaction step (c) is carried out in a solvent selected from water, polar organic solvent such as alcohols selected from methanol, ethanol, isopropanol, n-propanol, tert-butanol, n-butanol and the like or mixture there of.

The said step (c) is carried out at 0° to 70° C. and more preferably at 20° to 60° C.

The oxidation in step (d) is carried out by using the commonly known oxidising methods or agents such as Corey- Kim oxidation method, Dess- Martins reagent, Pfitzner moffat method or modifications thereof or with dimethyl sulfoxide in presence of oxalyl chloride or phosphorous pentoxide or p-Toluene sulfonyl chloride or acetic anhydride or N-chlorosuccinimide. The oxidation can also be carried out by Manganese or chromium or selenium reagents, tert-amine oxides or by any above oxidant in presence of phase transfer catalyst.

The compound of formula (XIII) is then treated with alcohol to obtain Telithromycin of formula (I) and if desired it can be treated further to obtain pharmaceutically acceptable salt thereof, by conventional methods. Alcohol is selected from group comprising of methanol, ethanol, n-propanol, isopropanol, tert-butanol, n-butanol and the like or mixtures thereof. The preferred alcohol is methanol.

The reaction step (e) is carried out at a temperature of 0° to 100° C. and preferably at 20° to 70° C. This step can be optionally carried out in the presence of mineral acid selected from group comprising of hydrochloric acid, sulphuric acid and the like or mixtures thereof.

Another preferred embodiment of the present invention provides an alternative process for the preparation of Telithromycin of formula (I) or pharmaceutically acceptable salts thereof, comprises steps of,
(a) reacting compound of formula (IX) with compound of formula (A),

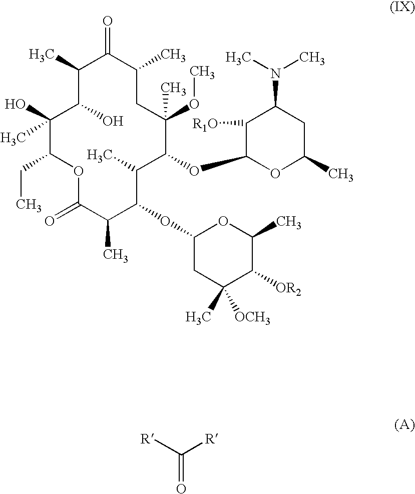

wherein R′ is selected from group comprising of methoxy, ethoxy, propoxy, pentamethylene, piperidinyl, 1-methyl imidazolyl and the like, in the presence of polar solvent and a base to obtain compound of formula (X),

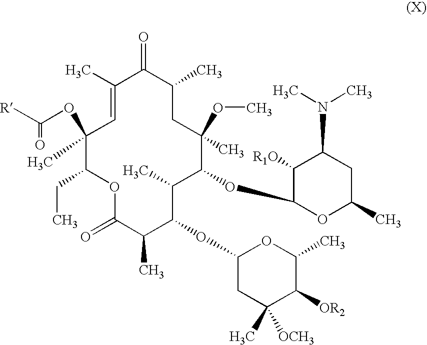

wherein R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴ and R⁵ is other than hydrogen atom) or

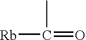

(wherein R_{b} is an alkyl group having 1 to 10 carbon atoms or a substituted or unsubstituted aryl)
(b) condensing compound of formula (X) with R—NH₂ in a suitable polar solvent to obtain compound of formula (XI)

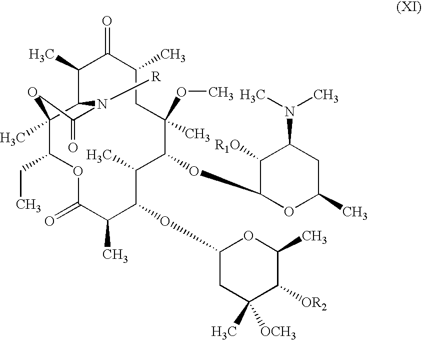

wherein R, R₁ and R₂ are same as defined hereinabove
(c) treating compound of formula (XI) with an acid to obtain compound of formula (XII)

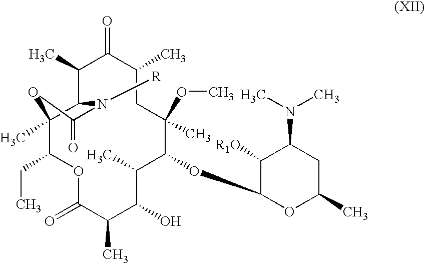

(f) treating compound of formula (XII) with an alcohol to obtain compound of formula (XIV)

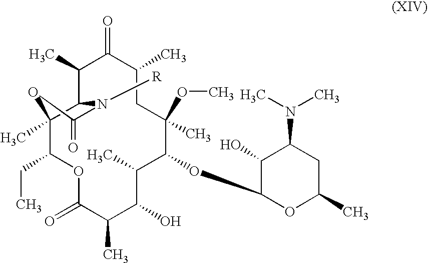

(g) Selectively oxidizing compound of formula (XIV) in the presence of an oxidizing agent.

In the alternative process for the preparation of Telithromycin, the reaction step (f) is carried out in the presence of an alcohol to give compound of formula (XIV). The alcohol in step (f) is selected from group comprising of methanol, ethanol, n-propanol, isopropanol, tert-butanol, n-butanol and the like or mixtures thereof. The preferred alcohol is methanol.

The reaction step (f) is carried out at a temperature of 0 to 100° C. and preferably at 20 to 70° C. This step can be optionally carried out in the presence of mineral acid selected from group comprising of hydrochloric acid, sulphuric acid and the like or mixtures thereof.

The compound of formula (XIV) is selectively oxidized to give Telithromycin of formula (I) and if desired it can be treated further to obtain pharmaceutically acceptable salt thereof by conventional methods. The said oxidation is carried out using Corey- Kim oxidation method, Dess- Martins reagent, Pfitzner moffat method or modifications thereof or with dimethyl sulfoxide in presence of oxalyl chloride or phosphorous pentoxide or p-Toluene sulfonyl chloride or acetic anhydride or N-chlorosuccinimide.

Further embodiment of the present invention provides a process for the preparation of novel compound of formula (X).

The process for the preparation of novel compound of formula (X) comprises steps of reacting compound of formula (IX) with compound of formula (A),

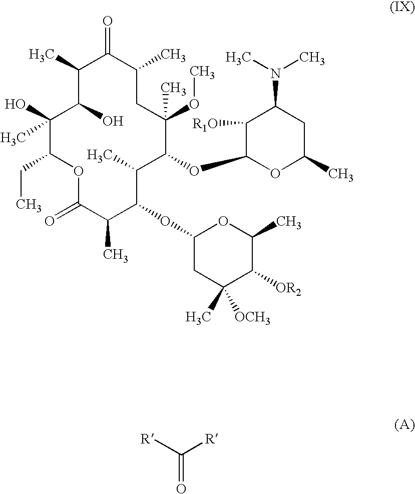

wherein R′ is selected from group comprising of methoxy, ethoxy, propoxy, pentamethylene, piperidinyl, 1-methyl imidazolyl and the like, in the presence of polar solvent and a base to obtain compound of formula (X)

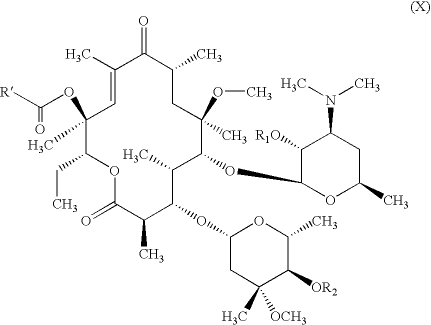

wherein R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴ and R⁵ is other than hydrogen atom) or

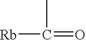

(wherein R_{b} is an alkyl group having 1 to 10 carbon atoms or a substituted or unsubstituted aryl)

The process of the present invention is described by the following examples, which are illustrative only and should not be construed so as to limit the scope of the invention in any manner.

### EXAMPLES

### Example 1

### Preparation of 2′,4″-di-O-bis(trimethylsilyl)-6-O-methylerythromycin A

20 g of Clarithromycin was dissolved in 200 ml DMF, 1.5 g ammonium chroride was added and 14.1 ml 1,1,1,3,3,3-hexamethyldisilazane was added dropwise at room temperature, and the mixture was stirred as such for 3 hours. After completion of the reaction water was poured into the reaction mixture and it was extracted with ethyl acetate. The organic layer was washed with saturated brine solution and dried over sodium sulphate. After distilling off ethyl acetate under vacuum, product was obtained which was crystallized from acetonitrile to give 23.5 g of title compound.

### Example 2

### Preparation of 10,11-anhydro-12-O-(1-methylimidazolylcarbonyl)-2′,4″-O-bis(trimethylsilyl)-6-O-methylerythromycin A

100 ml Dimethylformamide is added to 20 g of the compound obtained in example 1 at room temperature. 6.4 g DBU (1,8-Diazabicyclo[5.4.0]undec-7-ene) was added to the reaction mixture and stirred at room temperature. Further, 18 g 1,1′-Carbonylbisdiimidazole was added to the reaction mass and it was stirred till the reaction was complete. Water was added to obtain title compound (21 g).

### Example 3

### Preparation of 2′,4″-di-O-benzoyl-6-O-methylerythromycin A

1250 ml of ethyl acetate was added to 250 gm Clarithromycin A. 264.65 g benzoic anhydride, 57.20 g 4-dimethylamino pyridine and 67.60 g tri ethyl amine was added to the reaction mixture at 25° C. to 35° C. The reaction mixture was stirred for about 70 hours at ambient temperature After the completion of reaction, ethyl acetate was distilled out to obtain 2′,4″-di-O-benzoyl-6-O-methylerythromycin A

### Example 4

### Preparation of 10,11-anhydro-2′,4″-di-O-benzoyl-12-O-(1-methylimidazolyl carbonyl)-6-O-methylerythromycin A

100 ml Dimethylformamide is added to 2′,4″-di-O-benzoyl-6-O-methylerythromycin A at 25° C. to 35° C. 6.4 g DBU (1,8-Diazabicyclo[5.4.0]undec-7-ene) was added to the reaction mixture and stirred at ambient temperature. Further, 18 g 1,1′-Carbonylbisimidazole was added to the reaction mass and it was stirred until completion of reaction at ambient temperature. The desired compound is isolated by addition of water

### Example 5

### Preparation of 2′,4″-di-O— bis(trimethylsilyl)-11-amino-11-N-[4-[4-(3-pyridyl)imidazol-1-yl]butyl]-11-deoxy-6-O-methylerythromycin A 11,12-cyclic carbamate

100 ml dimethylformamide was added to 20 g of compound obtained in example 2 or 4 at room temperature. 6.3 g of 4-[4-(3-pyridyl)imidazol-1-yl]butylamine and 3 g of DBU was added to the reaction mixture and it was stirred at room temperature till the reaction was complete. The reaction mass was further treated with cold water and to obtain solid, which was treated with dichloromethane followed by extraction and removal of solvent to give title compound.

### Example 6

### Preparation of 2′-O— trimethylsilyl-11-amino-11-N-[4-[4-(3-pyridyl)imidazol-1-yl]butyl]-11-deoxy-5-O-desosaminyl-6-O-methylerythronolide A 11,12-cyclic carbamate

40 ml acetone was added to 20 g of compound obtained in example 3 to obtain clear solution at room temperature. Dilute hydrochloric acid (40 ml) was added to the reaction mixture and it was stirred for 24 hours at room temperature. After the completion of the reaction, the reaction mixture was extracted with ethyl acetate and treated with Sodium hydroxide solution to give title compound.

### Example 7

### Preparation of 2′-trimethylsilyl-11-amino-11-N-[4-[4-(3-pyridyl)imidazol-1-yl]butyl]-11-deoxy-5-O-desosaminyl-6-O-methylerythronolide A 11,12-cyclic carbamate

180 ml dichloromethane was added to 8.0 g N-chlorosuccinimide under nitrogen at room temperature cooled to 0° C. 7.2 ml dimethylsulfide was added slowly to the reaction mixture at 0° C. under stirring. 20 g compound obtained in example 4 was dissolved in 80 ml dichloromethane and was added drop wise to the reaction mixture at 0° C. under stirring. Further it was cooled to about −20° C. and solution of 16 ml Triethylamine in 20 ml dichloromethane is added to the reaction mixture and stirred for 30 minutes. After completion of the reaction, it is treated with saturated sodium bicarbonate solution and organic layer separated out. The organic layer was distilled out to obtain title compound.

### Example 8

### Preparation of Telithromycin

100 ml methanol was added to 10 g of compound obtained in example 5 at room temperature and the reaction mixture was heated to reflux for about 7 hours. After completion of the reaction, methanol was distilled off under vacuum at 45° C. 100 ml dilute hydrochloric acid was added to the residue and the aqueous layer was extracted with ethyl acetate and sodium bicarbonate and organic layer separated out. The product is obtained by distillation of solvent from organic layer and recrystallized from dichloromethane and metyl tert- butyl ether (MTBE) to give 9 g Telithromycin (yield: 98.9%, purity by HPLC: 99.23%)

### Example 9

### 2′-hydroxy-11-amino-11-N-[4-[4-(3-pyridyl) imidazol-1-yl]butyl]-11-deoxy-5-O-desosaminyl-6-O-methylerythronolide A 11,12-cyclic carbamate

10 g of compound obtained in example 4 was dissolved in 100 ml Isoproapnol and stirred at reflux temperature for 18 hours. The solvent was then distilled off under vacuum to give crude product as white foam. Then, the crude product was purified by refluxing in 20 ml of acetone and followed by 1 hour stirring at 10° C. The solution and filtered and the solid was washed with chilled acetone to give title compound.

### Example 10

### Preparation of Telithromycin

10 g of compound obtained in example 7 was dissolved in 200 ml dichloromethane and 15 g of Dess Martin reagent was added in one lot. The mixture was stirred at ambient temperature for 30 min. Further, 260 ml of saturated sodium bicarbonate solution is added and the mixture is stirred for 30 min. The solid precipitate is filtered and the organic layer is separated. The organic layer is washed with water, dried over sodium sulfate and solvent is distilled off under vacuum to give solid. Further, the solid was stirred with 40 ml of diisoproyl ether, filtered and dried to give 9 g Telithromycin (yield: 90%, purity by HPLC: 99.44%)

### Example 11

### Preparation of 10,11-anhydro-12-O-piperidinylcarbonyl)-2′,4″-O-bis(trimethylsilyl)-6-O-methylerythromycin A

100 ml Dimethylformamide is added to 20 g of the compound obtained in example 1 at room temperature. 6.4 g DBU (1,8-Diazabicyclo[5.4.0]undec-7-ene) was added to the reaction mixture and stirred at room temperature. Further, 17.5 g 1,1′-Carbonyldipiperidine was added to the reaction mass and it was stirred till the reaction was complete. Water was added to obtain title compound (19 g).

While the present invention has been described in terms of its specific embodiments, certain modifications and equivalents will be apparent to those skilled in the art and are intended to be included within the scope of the present invention.

1. A process for the preparation of compound of formula (I) (Telithromycin) or pharmaceutically acceptable salts thereof 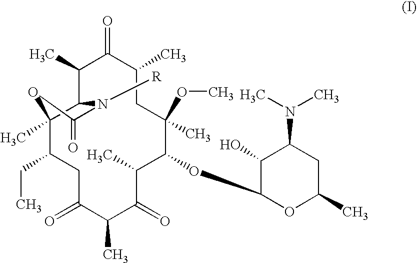
wherein R is 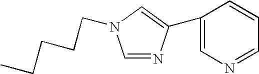
comprises steps of,
(a) reacting compound of formula (IX) with compound of formula (A), 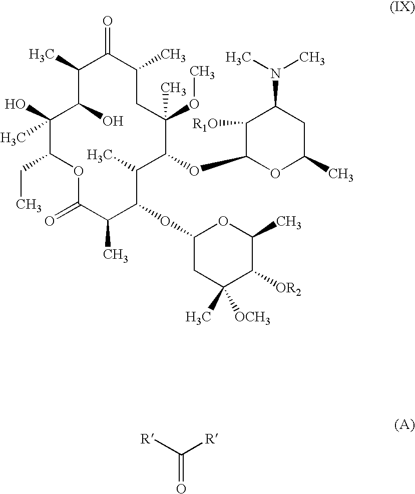
wherein R′ is selected from group comprising of methoxy, ethoxy, propoxy, pentamethylene, piperidinyl and 1-methyl imidazolyl, in the presence of a polar solvent and a base to obtain compound of formula (X) 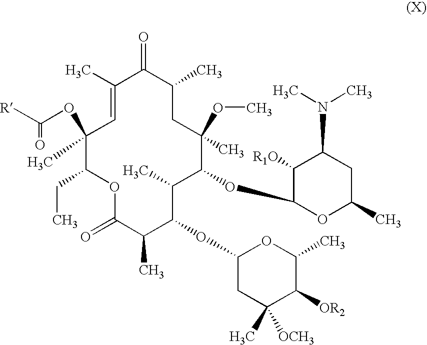
wherein R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴and R⁵ is other than hydrogen atom) or 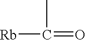
(wherein R_{b} is an alkyl group having 1 to 10 carbon atoms or a substituted or unsubstituted aryl)
condensing compound of formula (X) with R—NH₂ in a suitable polar solvent to obtain compound of formula (XI) 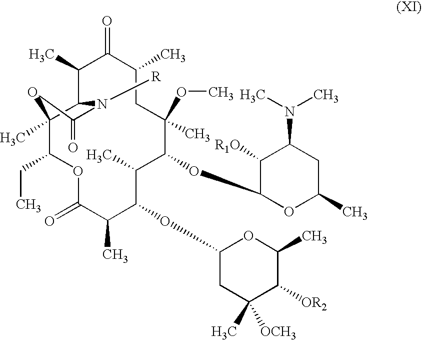
wherein R, R₁ and R₂ are same as defined hereinabove treating compound formula (XI) with an acid to obtain compound of formula (XII) 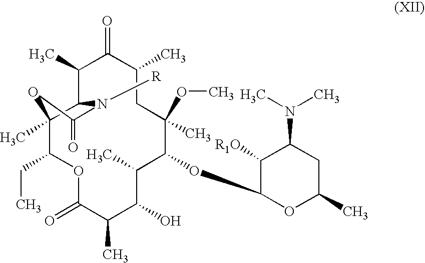
oxidizing compound of formula (XII) in the presence of oxidizing agent to form compound of formula (XIII) 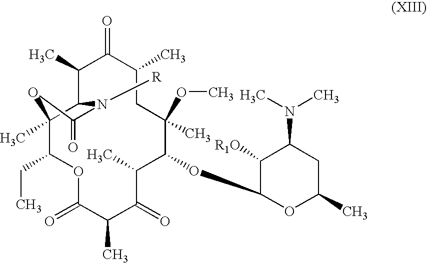
treating compound of formula (XIII) with an alcohol

2. A process for the preparation of compound of formula (I) or pharmaceutically acceptable salts thereof 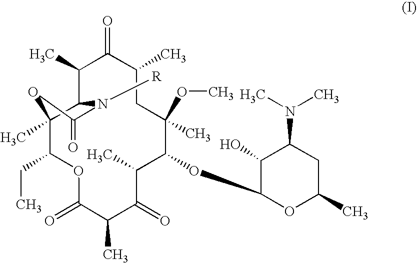
wherein R is 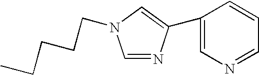
comprises steps of
(a) reacting compound of formula (IX) with compound of formula (A), 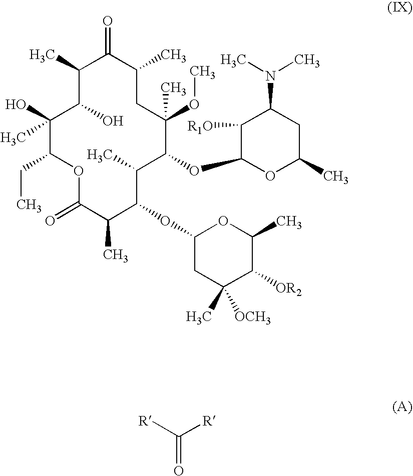
wherein R′ is selected from group comprising of methoxy, ethoxy, propoxy, pentamethylene, piperidinyl and 1-methyl imidazolyl, in the presence of a polar solvent and a base to obtain compound of formula (X), 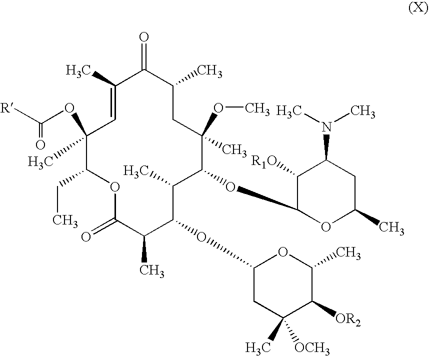
wherein R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴and R⁵ is other than hydrogen atom) or 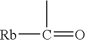
(wherein R_{b} is an alkyl group having 1 to 10 carbon atoms or a substituted or unsubstituted aryl)
condensing compound of formula (X) with R—NH₂ in a suitable polar solvent to obtain compound of formula (XI) 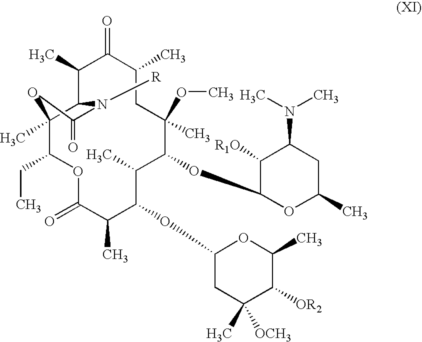
wherein R, R₁ and R₂ are same as defined hereinabove treating compound of formula (XI) with an acid to obtain compound of formula (XII) 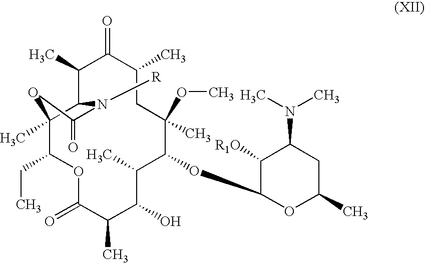
(f) treating compound of formula (XII) with an alcohol to obtain compound of formula (XIV) 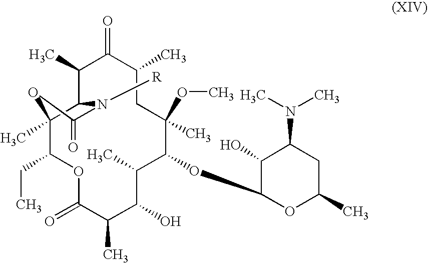
selectively oxidizing compound of formula (XIV) in the presence of an oxidizing agent.

3. A process as claimed in claim 1 and 2, wherein said compound of formula (A) is selected from group comprising of 1,1′-carbonylbis(2-methylimidazole), 1,1′-carbonyldipiperidine, bis(pentamethylene)urea, dimethylcarbonyl, diethoxycarbonyl and dipropoxycarbonyl.

4. A process as claimed in claim 1, wherein said polar solvent in step (a) is selected from dimethylformamide, tetrahydrofuran, acetonitrile and the like or mixtures thereof.

5. A process as claimed in claim 1, wherein said base in step (a) is selected from DBU, triethylamine and diisopropylethylamine.

6. A process as claimed in claim 1, wherein said protecting group is selected from group comprising of trimethylsilyl group, a triethylsilyl group, an isopropyldimethylsilyl group, a tert-butyldimethylsilyl group, a (triphenylmethyl)dimethylsilyl group, a tert-butyldiphenylsilyl group, a diphenylmethylsilyl group, diphenylvinylsilyl group, a methyldiisopropylsilyl group, a tribenzylsilyl group, a tri(p-xylyl)silyl group, a triphenylsilyl group, a diphenylsilyl group and a dimethyloctadecylsilyl group, a acetyl group, a benzyloxy carbonyl group or a benzoyl group

7. A process as claimed in claim 1 and 2, wherein said polar solvent in step (b) is selected from group comprising of methanol, ethanol, isopropanol, n-propanol, n-butanol, iso butyl alcohol, tert-butyl alcohol, methoxyethanol, ethoxyethanol, pentanol, neo-pentyl alcohol, tert-pentyl alcohol, cyclohexanol, ethylene glycol, propylene glycol, benzyl alcohol, phenol, glycerol, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethylsulfoxide, propionitrile, ethyl formate, methyl acetate, hexachloroacetone, HMPA, HMPT, acetone, ethyl methyl ketone, ethyl acetate, isopropyl acetate, t-butyl acetate, sulfolane, N,N-dimethylpropionamide, nitromethane, nitrobenzene, tetrahydrofuran (THF), dioxane, water, polyethers or mixtures thereof.

8. A process as claimed in claim 7, wherein said polar solvent is selected from dimethylformamide and acetonitrile.

9. A process as claimed in claim 1, wherein said step (b) is optionally carried out in the presence of base selected from DBU, triethylamine and diisopropylethylamine

10. A process as claimed in claim 1, wherein said acid in step (c) is selected from organic or inorganic acid.

11. A process as claimed in claim 10, wherein said acid is selected from group comprising of hydrochloric acid, hydrobromic acid, sulfuric acid, nitric acid, phosphoric acid, perchloric acid or hydrofluoric acid.

12. A process as claimed in claim 1, wherein step (c) is carried out in a solvent selected from group comprising of water, polar organic solvents or mixtures thereof.

13. A process as claimed in claim 12, wherein said solvent is selected from group comprising of water, alcohol or mixtures thereof.

14. A process as claimed in claim 13, wherein said solvent is selected from group comprising of water, methanol, ethanol, isopropanol, n-propanol, tert-butanol, n-butanol or mixtures thereof.

15. A process as claimed in claim 1, wherein said oxidation in step (d) is carried out using Corey- Kim oxidation method, Dess- Martin reagent, Pfitzner Moffat method or modifications thereof or with dimethyl sulfoxide in presence of oxalyl chloride or phosphorous pentoxide or p-Toluene sulfonyl chloride or acetic anhydride or N-chlorosuccinimide or by manganese or chromium or selenium reagents, tert-amine oxides or any said oxidant in presence or absence of phase transfer catalyst.

16. A process as claimed in claim 1, wherein said alcohol in step (e) is selected from group comprising of methanol, ethanol, n-propanol, iso propanol, tert-butanol, n-butanol or mixtures thereof.

17. A process as claimed in claim 1, wherein step (e) is optionally carried out in the presence of mineral acid selected from hydrochloric acid, sulphuric acid or mixtures thereof.

18. A process as claimed in claim 2, wherein said alcohol in step (f) is selected from group comprising of methanol, ethanol, n-propanol, iso propanol, tert-butanol, n-butanol or mixtures thereof.

19. A process as claimed in claim 2, wherein step (f) is optionally carried out in the presence of mineral acid selected from hydrochloric acid, sulphuric acid or mixtures thereof.

20. A process as claimed in claim 2, wherein said oxidation in step (g) is carried out using Corey- Kim oxidation method, Des- Martins reagent, Pfitzner moffat method or modifications thereof or with dimethyl sulfoxide in presence of oxalyl chloride or phosphorous pentoxide or p-Toluene sulfonyl chloride or acetic anhydride or N-chlorosuccinimide.

21. The novel compound of formula (X) 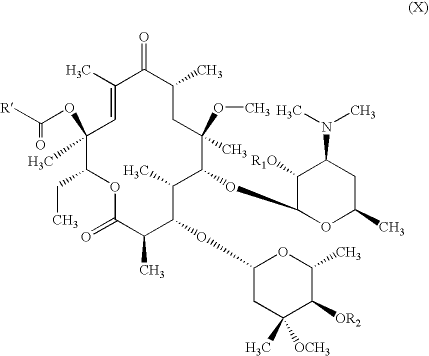
wherein R′ is selected from comprising group comprising of methoxy, ethoxy, propoxy, pentamethylene, piperidinyl and 1-methyl imidazolyl and R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴ and R⁵ is other than hydrogen atom) or Rb—C═O (wherein R_{b} is an alkyl group having 1 to 10 carbon atoms or a substituted or unsubstituted aryl)

22. A process for the preparation of novel compound of formula (X) comprising, reacting compound of formula (IX) with compound of formula (A), 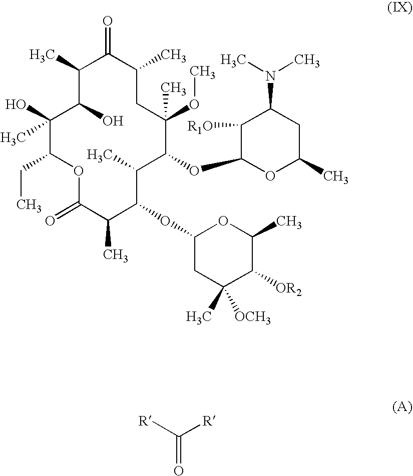
wherein R′ is selected from group comprising of methoxy, ethoxy, propoxy, pentamethylene, piperidinyl andl-methyl imidazolyl in the presence of polar solvent and a base to obtain compound of formula (X) 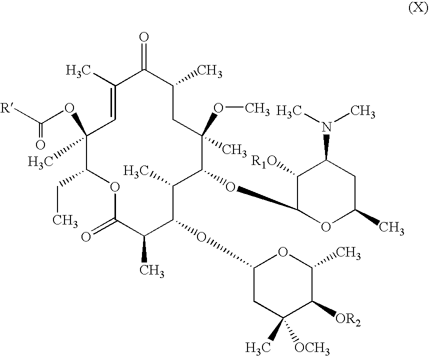
wherein R₁ and R₂ are same or different protecting groups selected from group comprising of substituted silyl group of formula —SiR³R⁴R⁵ (wherein R³, R⁴ and R⁵ are the same or different, and each is a hydrogen atom, an alkyl group having 1 to 15 carbon atoms, a phenyl substituted alkyl group in which the alkyl moiety has 1 to 3 carbon atoms, a phenyl group, a cycloalkyl group having 5 to 7 carbon atoms or an alkenyl group having 2 to 5 carbon atoms, with the proviso that at least one of R³, R⁴and R⁵ is other than hydrogen atom) or 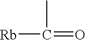
(wherein R_{b} is an alkyl group having 1 to 10 carbon atoms or a substituted or unsubstituted aryl).

23. A process as claimed in claim 23, wherein said compound of formula (A) is selected from group comprising of 1,1′-carbonylbis(2-methylimidazole), 1,1′-carbonyldipiperidine, bis(pentamethylene)urea, dimethylcarbonyl, diethoxycarbonyl and dipropoxycarbonyl.

24. A process as claimed in claim 23, wherein said protecting group is selected from group comprising of trimethylsilyl group, a triethylsilyl group, an isopropyldimethylsilyl group, a tert-butyldimethylsilyl group, a (triphenylmethyl)dimethylsilyl group, a tert-butyldiphenylsilyl group, a diphenylmethylsilyl group, diphenylvinylsilyl group, a methyldiisopropylsilyl group, a tribenzylsilyl group, a tri(p-xylyl)silyl group, a triphenylsilyl group, a diphenylsilyl group and a dimethyloctadecylsilyl group, a acetyl group, a benzyloxy carbonyl group or a benzoyl group.

25. A process as claimed in claim 2, wherein said compound of formula (A) is selected from group comprising of 1,1′-carbonylbis(2-methylimidazole), 1,1′-carbonyldipiperidine, bis(pentamethylene)urea, dimethylcarbonyl, diethoxycarbonyl and dipropoxycarbonyl.

26. A process as claimed in claim 2, wherein said polar solvent in step (a) is selected from dimethylformamide, tetrahydrofuran, acetonitrile and the like or mixtures thereof.

27. A process as claimed in claim 2, wherein said base in step (a) is selected from DBU, triethylamine and diisopropylethylamine.

28. A process as claimed in claim 2, wherein said protecting group is selected from group comprising of trimethylsilyl group, a triethylsilyl group, an isopropyldimethylsilyl group, a tert-butyldimethylsilyl group, a (triphenylmethyl)dimethylsilyl group, a tert-butyldiphenylsilyl group, a diphenylmethylsilyl group, diphenylvinylsilyl group, a methyldiisopropylsilyl group, a tribenzylsilyl group, a tri(p-xylyl)silyl group, a triphenylsilyl group, a diphenylsilyl group and a dimethyloctadecylsilyl group, a acetyl group, a benzyloxy carbonyl group or a benzoyl group.

29. A process as claimed in claim 2, wherein said polar solvent in step (b) is selected from group comprising of methanol, ethanol, isopropanol, n-propanol, n-butanol, iso butyl alcohol, tert-butyl alcohol, methoxyethanol, ethoxyethanol, pentanol, neo-pentyl alcohol, tert-pentyl alcohol, cyclohexanol, ethylene glycol, propylene glycol, benzyl alcohol, phenol, glycerol, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethylsulfoxide, propionitrile, ethyl formate, methyl acetate, hexachloroacetone, HMPA, HMPT, acetone, ethyl methyl ketone, ethyl acetate, isopropyl acetate, t-butyl acetate, sulfolane, N,N-dimethylpropionamide, nitromethane, nitrobenzene, tetrahydrofuran (THF), dioxane, water, polyethers or mixtures thereof.

30. A process as claimed in claim 29, wherein said polar solvent is selected from dimethylformamide and acetonitrile.

31. A process as claimed in claim 2, wherein said step (b) is optionally carried out in the presence of base selected from DBU, triethylamine and diisopropylethylamine.

32. A process as claimed in claim 2, wherein said acid in step (c) is selected from organic or inorganic acid.

33. A process as claimed in claim 2, wherein step (c) is carried out in a solvent selected from group comprising of water, polar organic solvents or mixtures thereof.
